(19) United States
(12) Patent Application Publication
(10) Pub. No.: US 2008/0038301 A1
(43) Pub. Date: Feb. 14, 2008

(54) Personal care product comprising collapsible water-containing capsules

(75) Inventor: Kanna Ueda, Kobe (JP)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/890,364

(22) Filed: Aug. 6, 2007

Related U.S. Application Data
(60) Provisional application No. 60/836,430, filed on Aug. 8, 2006.
(60) Provisional application No. 60/933,576, filed on Jun. 7, 2007.

(51) Int. Cl. A61K 8/02; A61K 33/00; A61K 9/48; A61P 17/00

(52) U.S. Cl. 424/401; 424/455; 424/600

(57) ABSTRACT

A personal care product comprising: (a) a collapsible water-containing capsule having water encapsulated or dispersed in a first pigment component which is a hydrophobic pigment having a particle size of less than 1 μm; and (b) an applicator for applying the capsule on a personal care surface, wherein the surface of the applicator is made of material having open cells.

### CROSS REFERENCE TO RELATED APPLICATIONS

This application claims the benefit of U.S. Provisional Application No. 60/836,430 filed on Aug. 8, 2006; and U.S. Provisional Application No. 60/933,576 filed on Jun. 7, 2007.

### FIELD OF THE INVENTION

The present invention relates to a personal care product comprising a collapsible water-containing capsule having water encapsulated or dispersed in hydrophobic pigments; and an applicator for applying the capsule on a personal care surface, wherein the surface of the applicator is made of material having open cells. The present invention further relates to methods of make-up of the skin using such personal care product.

### BACKGROUND

A foundation composition can be applied to the face and other parts of the body to even skin tone and texture and to hide pores, imperfections, fine lines and the like. A foundation composition is also applied to moisturize the skin, to balance the oil level of the skin, and to provide protection against the adverse effects of sunlight, wind, and other environmental factors.

Foundation compositions are generally available in the form of liquid or cream suspensions, emulsions, gels, pressed powders, loose powders or anhydrous oil and wax compositions. Emulsion-type foundations are suitable in that they provide moisturizing effects by the water and water-soluble skin treatment agents incorporated. On the other hand, a larger amount and variation of powders and pigments can be formulated into pressed powders and loose powders.

Recently, consumers who seek moisturization as well as the ideal look having both good coverage and natural look on the skin, have the habit of a two step regimen of foundation application. The two step regimen typically contains application of a liquid or emulsion form foundation followed by a pressed or loose powder foundation. It is conceived by such demanding consumers that such two-step regimen provides best results, however, such regimen is also quite elaborate. There is a need for a foundation product which can provide both good feel and good appearance on the skin, and also being easy to apply on the skin.

Meanwhile, collapsible water-containing capsules are known in the art, such as in WO 01/85138, Japanese Patent Publications 2001-131528A, 2000-247823A, 2000-309506A, 11-130614A, 10-265367A, 5-65212A, 4-308520A, 2006-509732A, 2001-226230A, 2001-158716A, and 1-125314A. Such capsules provide a unique feel or change of feel upon application and collapsing on the skin. Upon application to the skin, such capsules provide a moisturizing or fresh feeling. Such capsules may also deliver water-soluble skin active agents such as vitamin C derivatives to the skin, in a more or less stable manner. The collapsible water-containing capsules form may be adopted for foundations having such benefits.

None of the above mentioned references, however, disclose an applicator which allows suitable and easy application of the mentioned capsules on the skin, nor method of applying to the skin. Further, capsule products available on the market are sold with applicators such as brushes and puffs, the puffs having an outer layer of polyester cloth. Such applicators on the market are not suitable for applying capsules in a even manner on the skin.

Based on the foregoing, there is a need for a personal care product comprising a collapsible water-containing capsule which provides good feel to the personal surface, and an applicator suitable for applying such collapsible water-containing capsule on the personal surface. While such benefit is believed to be useful for foundations, it is also believed that the same is true for other personal care compositions.

None of the existing art provides all of the advantages and benefits of the present invention.

### SUMMARY

The present invention is directed to a personal care product comprising:
(a) a collapsible water-containing capsule having water encapsulated or dispersed in a first pigment component which is a hydrophobic pigment having a particle size of less than 1 μm; and
(b) an applicator for applying the capsule on a personal care surface, wherein the surface of the applicator is made of material having open cells.

The present invention is also directed to a method of treating or making up the skin utilizing the aforementioned collapsible water-containing capsule.

These and other features, aspects, and advantages of the present invention will become evident to those skilled in the art from a reading of the present disclosure with the appended claims.

### BRIEF DESCRIPTION OF THE DRAWINGS

The patent or application file contains at least one drawing executed in color. Copies of this patent or patent application publication with color drawings will be provided by the Office upon request and payment of the necessary fee.

While the specification concludes with claims particularly pointing out and distinctly claiming the invention, it is believed that the present invention will be better understood from the following description of preferred, nonlimiting embodiments and representations taken in conjunction with the accompanying drawings in which:

FIG. 1 is an enlarged photograph of a preferred embodiment material, HP-70 made of polyurethane provided by Bridgestone, suitable for the applicator of the present invention, each scale being the length of 5 mm.

FIG. 1 is an enlarged photograph of an embodiment material, Max Factor Profinish Buff made of nitrile butadiene rubber provided by Max Factor K.K., less suitable for the applicator of the present invention, each scale being the length of 5 mm.

### DETAILED DESCRIPTION

While the specification concludes with claims particularly pointing out and distinctly claiming the invention, it is believed that the present invention will be better understood from the following description.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

All ingredients such as actives and other ingredients useful herein may be categorized or described by their cosmetic and/or therapeutic benefit or their postulated mode of action. However, it is to be understood that the active and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed.

### Applicator

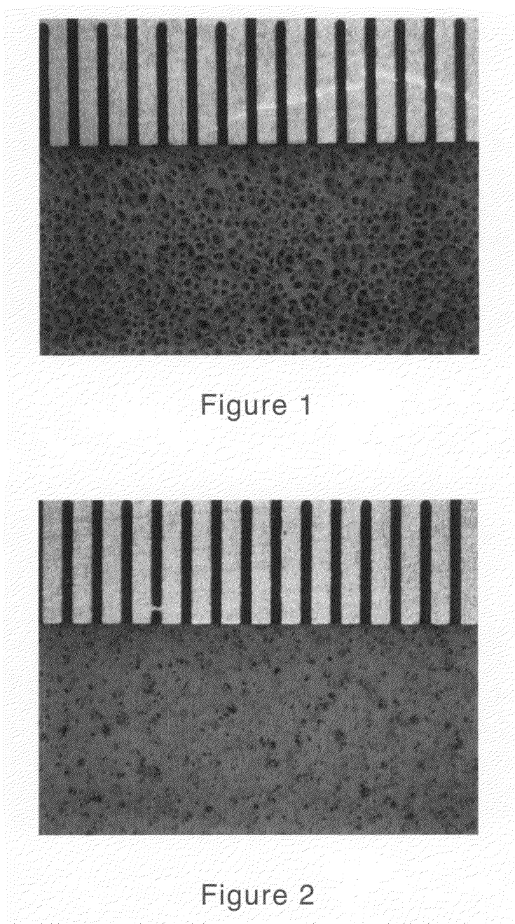

The personal care product of the present invention comprises an applicator for applying the collapsible water-containing capsule on a personal care surface, particularly skin. The applicator is made of material having an open cell. It is well known in the art that sponge-like articles can be made of various materials via different processes, and that the resulting article may have open cells or closed cells. Materials made of open cells useful for personal care use may be represented by polyurethane, as shown in FIG. 1. The open cells provide more or less of an open structure with the surrounding cells, and provide a kind of reservoir within the structure capable of holding solid and liquid substances. Materials made of closed cells useful for personal care use may be represented by NBR (nitrile butadiene rubber), as shown in FIG. 2. NBR is a material commonly used for cosmetic foundation products. The closed cells are more or less independent, and are less capable of holding solid and liquid substances.

Surprisingly, it has been found that, applicators made by open cells are easier for applying collapsible water-containing capsules on the skin, and provide improved feel, compared to applicators made by closed cells. For capsules designed to provide appearance benefits, surprisingly, it has been found that, applicators made by open cells also provide improved appearance on the skin, compared to applicators made by closed cells.

Without being bound by theory, it is believed that, upon pay off of the product, the open cell materials act as a temporary reservoir to hold some of the capsules within the structure of the cells. Thus, a more or less regulated amount of the capsules are applied on the skin via a number of strokes on the skin. By such more or less controlled application of the capsules, the capsules can be evenly applied on the skin. Further, in that more or less regulated amount of capsules are collapsed on the skin upon each stroke, the freshening, cooling, watery, or moisturizing feel caused by water release can be felt for a prolonged length of time. For capsules designed to provide appearance benefits, it is believed that the more or less regulated collapsing also leads to more even application of the pigments provided by the collapsed capsule, thereby providing improved appearance.

Further, without being bound by theory, it is believed that such benefits are less observed with closed cell materials, in that the capsules are hardly held within the structure of the cells, and thus a greater amount of capsules collapse in a single stroke.

Based on the foregoing, the surface of the applicator of the present invention is made of material having open cells to serve as a temporary reservoir for the capsules. So long as a sufficient thickness of the applicator is made of material having open cells to provide such reservoir capability, the remainder of the applicator may be made with other materials. For example, the core of the applicator may be made by other material for providing an overall rigidity, resiliency, or a particular shape to the applicator. Further, the applicator may further comprise raised projections, the raised projections attached in a manner such that the surface of the material having open cells continues to serve as a temporary reservoir of the capsules. Such raised portions may be attached to the applicator for providing an additional skin feel upon application.

Preferably, the present applicator is made of polyurethane. Preferably, the polyurethane material is treated to remove foam films. Preferably, the present applicator has an average cell range of from about 40/25 mm to about 80/25 mm, more preferably from about from about 60/25 mm to about 70/25 mm. The unit “number/25 mm” is for counting the number of cells found within the length of 25 mm, and is commonly used in the art. The greater the count, the finer the cells. The open cells of the present invention have a size suitable for providing the reservoir capability of the capsules. Depending on the size and affinity of the capsules, the cell size and material may be selected.

Commercially available materials useful herein include: polyurethanes with codes HP-70, HF-55, HW-80, HZ-80 and HX-80, all available from Bridgestone Corporation.

### Collapsible Water-containing Capsule

The personal care product of the present invention comprises a collapsible water-containing capsule which comprises, by weight of the capsule, at least about 60%, preferably from about 70% to about 92% of a water phase, the water phase being water and optional water-soluble solvents. To hold such abundant amount of water in the structure, the capsule of the present invention comprises a first pigment component which is a hydrophobic pigment having a particle size of less than 1 μm, in which the water is encapsulated or dispersed. The present capsule is preferably stable under normal storage conditions as well as normal mixing processes, however, collapses upon application.

In one preferred embodiment, such stability for the capsule is provided by comprising, in the present capsule, a gelling agent, a first pigment component, and a second component. Without being bound by theory, it is believed that the gelling agent holds the water phase in a relatively rigid structure, while the first and second pigment components cover the water phase and thereby provide the stability and integrity of the capsule.

In such preferred embodiment, preferably, the capsule of the present invention is substantially free of surfactant. Without being bound by theory, in such preferred embodiment, it is believed that surfactants may negatively affect the stability and shear stress tolerance of the present capsule by decreasing the surface tension difference between the water phase and the first and second pigment components. Herein, surfactants include those which have detersive capability, as well as those which only act as emulsifiers for emulsifying water and oil phases.

In such preferred embodiment, preferably, the capsule of the present invention is substantially free of porous pigments having a particle size of less than 1 μm. Without being bound by theory, in such preferred embodiment, it is believed that porous pigments of small size may absorb sebum from the personal surface to such an extent that a dry negative feeling is provided to the personal surface. Herein, porous pigments include silica, aluminum oxide, calcium carbonate, cellulose, and others that may have a porous structure when observed under magnification. It is noted that pigments made from the same chemical compound may take either a porous or non-porous structure, based on the process it is purified, processed, synthesized, or otherwise treated.

The capsule of the present invention provides unique benefits on the personal surface, such as skin, hair, or scalp, when collapsed on the surface. It provides a fresh or cooling feel to the surface, by releasing the abundant water. In the preferred embodiment mentioned above, the capsule provides an initially fresh, and then moisturizing feel to the surface. The preferred embodiment capsule further provides a good feel to the surface by the characteristic of the first and/or second pigment components. When the first and second pigment components are applied on the surface, the components provide the appearance benefits inherent of such pigment components.

The capsule of the present invention may, by itself, provide a product in the form of a loose powder product. The capsule of the present invention may also be mixed with other components to provide different product forms.

The collapsible water-containing capsule of the present invention is particularly useful for personal care compositions for delivering water, the pigments, and other components to personal surface. Personal care compositions herein include those for the purpose of skin care, make-up, extensive treatment, perfume, antiperspiration, deodorizing, hair coloring, hair treatment, hair styling, and others. Personal care compositions herein can take the product form of powders, wax solidified solid forms, liquids, lotions, pastes, aerosols, and others. One highly preferred product form embodiment is powder for use on the skin, such as foundation and skin care products.

In one highly preferred embodiment, the capsule comprises by weight:
(a) from about 70% to about 92% of a water phase comprising, by weight of the capsule:

(1) from about 5% to about 91.9% water; and

(2) from about 0.1% to about 20% of a gelling agent;
(b) from about 1% to about 29.9% of a first pigment component which has a particle size of less than 1 μm and is surface coated with a lipophobic hydrophobic coating material; and
(c) from about 0.1% to about 29% of a second pigment component which has a particle size of 1 μm or more, is surface coated with a hydrophobic coating material; and is spherical in shape; wherein the total of the first pigment component and the second pigment component is at least about 8% of the capsule.

The capsule of the present invention is particularly suitable for incorporating in personal care compositions for use on the skin, for treatment of the skin, and make-up of the skin. Accordingly, the present invention is also related to a method of treating or making up of the skin comprising the steps of:
(1) providing the collapsible water-containing capsule;
(2) shearing the collapsible water-containing capsule on the skin by the applicator to allow the collapsible water-containing capsule to collapse; whereby the components of the collapsible water-containing capsule are applied on the skin; and
(3) allowing the water to evaporate and/or be absorbed in the skin.

The present invention is also related to a method of treating or making up on the skin using the preferred embodiment capsules mentioned above, comprising the steps of:
(1) providing the collapsible water-containing capsule of the present invention;
(2) shearing the collapsible water-containing capsule on the skin by a finger or an applicator to allow the collapsible water-containing capsule to collapse; whereby the components of the collapsible water-containing capsule are applied on the skin, preferably whereby the components are applied on the skin in the order of vicinity from the skin, water, the first pigment component, and the second pigment component; and
(3) allowing the water to evaporate and/or be absorbed in the skin.

For such personal skin care compositions, the first pigment component and optional second pigment component are selected to provide the appropriate skin treatment and/or make-up benefits.

### Water Phase

The capsule of the present invention comprises a water phase, the water phase comprising water, optional gelling agent, and optional water-soluble solvent detailed hereafter. The present capsule comprises, by weight of the capsule, at least from about 60%, preferably from about 70% to about 92% of the water phase. The water phase may be made only by water. Preferably, water is contained at from about 5% to about 91.9% of the capsule. Deionized water is preferably used. Water from natural sources including mineral cations can also be used, depending on the desired characteristic of the product. In one preferred embodiment, water may be sourced from fermented biological cultures or its filtrates. A highly preferred commercial source of this kind is Saccharomycopsis ferment filtrate by the tradename SK-II Pitera available from Kashiwayama.

The pH of the water phase is selected in view of the desired characteristic of the product, and particularly, when skin benefit agents are included, the activity and stability of the skin benefit agents. In one preferred embodiment the pH is adjusted from about 4 to about 8. Buffers and other pH adjusting agents can be included to achieve the desirable pH.

### Water-Soluble Solvent

The water phase of the capsule of the present invention may further comprise a water-soluble solvent selected from lower alkyl alcohols and water-soluble humectants. The water-soluble solvents are selected according to the desired skin feel to be delivered, and/or for delivering certain skin benefit agents.

Lower alkyl alcohols useful herein are monohydric alcohols having 1 to 6 carbons, more preferably ethanol and isopropanol.

Water soluble humectants useful herein include polyhydric alcohols such as butylene glycol(1,3-butanediol), pentylene glycol(1,2-pentanediol), glycerin, sorbitol, propylene glycol, hexylene glycol, ethoxylated glucose, 1,2-hexane diol, 1,2-pentane diol, hexanetriol, dipropylene glycol, erythritol, trehalose, diglycerin, xylitol, maltitol, maltose, glucose, fructose; and other water-soluble compounds such as urea, sodium chondroitin sulfate, sodium hyaluronate, sodium adenosin phosphate, sodium lactate, pyrrolidone carbonate, glucosamine, cyclodextrin, and mixtures thereof. Also useful herein include water soluble alkoxylated nonionic polymers such as polyethylene glycols and polypropylene glycols having a molecular weight of up to about 1000 such as those with CTFA names PEG-200, PEG-400, PEG-600, PEG-1000, and mixtures thereof.

In one preferred embodiment, the present capsule comprises from about 1% to about 30% of a water-soluble humectant. In one highly preferred embodiment wherein the capsule is used as a foundation, the capsule comprises from about 3% to about 30% of a water-soluble humectant.

Commercially available humectants herein include: butylene glycol with tradename 1,3-Butylene Glycol available from Celanese, pentylene glycol with tradename HYDROLITE-5 available from Dragoco, glycerin with tradenames STAR and SUPEROL available from The Procter & Gamble Company, CRODEROL GA7000 available from Croda Universal Ltd., PRECERIN series available from Unichema, and a same tradename as the chemical name available from NOF; propylene glycol with tradename LEXOL PG-865/855 available from Inolex, 1,2-PROPYLENE GLYCOL USP available from BASF; sorbitol with tradenames LIPONIC series available from Lipo, SORBO, ALEX, A-625, and A-641 available from ICI, and UNISWEET 70, UNISWEET CONC available from UPI; dipropylene glycol with the same tradename available from BASF; diglycerin with tradename DIGLYCEROL available from Solvay GmbH; xylitol with the same tradename available from Kyowa and Eizai; maltitol with tradename MALBIT available from Hayashibara, sodium chondroitin sulfate with the same tradename available from Freeman and Bioiberica, and with tradename ATOMERGIC SODIUM CHONDROITIN SULFATE available from Atomergic Chemetals; sodium hyaluronate available from Chisso Corp, the same with tradenames ACTIMOIST available from Active Organics, AVIAN SODIUM HYALURONATE series available from Intergen, HYALURONIC ACID Na available from Ichimaru Pharcos; sodium adenosin phophate with the same tradename available from Asahikasei, Kyowa, and Daiichi Seiyaku; sodium lactate with the same tradename available from Merck, Wako, and Showa Kako, cyclodextrin with tradenames CAVITRON available from American Maize, RHODOCAP series available from Rhone-Poulenc, and DEXPEARL available from Tomen; and polyethylene glycols with the tradename CARBOWAX series available from Union Carbide.

### Gelling Agents

The collapsible water-containing capsule of the present composition may further comprise, by weight of the capsule, from about 0.1% to about 20%, preferably from about 0.1% to about 5%, of a gelling agent that provides the water phase a viscosity of from about 10 mPas to about 1,000,000 mPas, preferably from about 10 mPas to about 100,000 mPas. Incorporation of a gelling agent is advantageous, in that the gelling agent holds water and optional water-soluble solvents in a relatively rigid structure, and thereby believed to provide improved stability and integrity of the capsule.

The polymers useful as the gelling agent herein are water soluble or water miscible polymers. The term “water soluble or water miscible” with regard to the gelling agents herein, relate to compounds that are dissolved to make a transparent solution when dissolved in ample amount of water with or without the aid of elevated temperature and/or mixing.

Useful herein are starch derivative polymers such as carboxymethyl starch, and methylhydroxypropyl starch. Commercially available compounds that are highly useful herein include sodium carboxymethyl starch with tradename COVAGEL available from LCW.

Useful herein are cellulose derivative polymers. Cellulose derivative polymers useful herein include methylcellulose, ethylcellulose, hydroxyethylcellulose, hydroxyethyl ethylcellulose, hydroxypropyl methyl cellulose, nitrocellulose, sodium cellulose sulfate, sodium carboxymethylcellulose, crystalline cellulose, cellulose powder, and mixtures thereof. Also useful are starch derivative polymers such as carboxymethyl starch, and methylhydroxypropyl starch. Commercially available compounds that are highly useful herein include hydroxyethylcellulose with tradename Natrosol Hydroxyethylcellulose, and carboxymethylcellulose with tradename Aqualon Cellulose Gum, both available from Aqualon.

Useful herein are carboxylic acid/carboxylate copolymers. Commercially available carboxylic acid/carboxylate copolymers useful herein include: CTFA name Acrylates/C10-30 Alkyl Acrylate Crosspolymer having tradenames Pemulen TR-1, Pemulen TR-2, Carbopol 1342, Carbopol 1382, and Carbopol ETD 2020, all available from B. F. Goodrich Company.

Neutralizing agents may be included to neutralize the carboxylic acid/carboxylate copolymers herein. Nonlimiting examples of such neutralizing agents include sodium hydroxide, potassium hydroxide, ammonium hydroxide, monoethanolamine, diethanolamine, triethanolamine, diisopropanolamine, aminomethylpropanol, tromethamine, tetrahydroxypropyl ethylenediamine, and mixtures thereof.

Polyalkylene glycols having a molecular weight of more than about 1000 are useful herein. Useful are those having the following general formula:

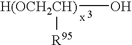

wherein R⁹⁵ is selected from the group consisting of H, methyl, and mixtures thereof. When R⁹⁵ is H, these materials are polymers of ethylene oxide, which are also known as polyethylene oxides, polyoxyethylenes, and polyethylene glycols. When R⁹⁵ is methyl, these materials are polymers of propylene oxide, which are also known as polypropylene oxides, polyoxypropylenes, and polypropylene glycols. When R⁹⁵ is methyl, it is also understood that various positional isomers of the resulting polymers can exist. In the above structure, x3 has an average value of from about 1500 to about 25,000, preferably from about 2500 to about 20,000, and more preferably from about 3500 to about 15,000. Other useful polymers include the polypropylene glycols and mixed polyethylene-polypropylene glycols, or polyoxyethylene-polyoxypropylene copolymer polymers. Polyethylene glycol polymers useful herein are PEG-2M wherein R⁹⁵ equals H and x3 has an average value of about 2,000 (PEG-2M is also known as Polyox WSR® N-10, which is available from Union Carbide and as PEG-2,000); PEG-5M wherein R⁹⁵ equals H and x3 has an average value of about 5,000 (PEG-5M is also known as Polyox WSR® N-35 and Polyox WSR® N-80, both available from Union Carbide and as PEG-5,000 and Polyethylene Glycol 300,000); PEG-7M wherein R equals H and x3 has an average value of about 7,000 (PEG-7M is also known as Polyox WSR® N-750 available from Union Carbide); PEG-9M wherein R⁹⁵ equals H and x3 has an average value of about 9,000 (PEG 9-M is also known as Polyox WSR® N-3333 available from Union Carbide); and PEG-14 M wherein R⁹⁵ equals H and x3 has an average value of about 14,000 (PEG-14M is also known as POLYOX WSR® N-3000 available from Union Carbide).

Useful herein are vinyl polymers such as cross linked acrylic acid polymers with the CTFA name Carbomer, pullulan, mannan, scleroglucans, polyvinylpyrrolidone, polyvinyl alcohol, guar gum, hydroxypropyl guar gum, xanthan gum, acacia gum, arabia gum, tragacanth, galactan, carob gum, karaya gum, locust bean gum, carrageenin, pectin, amylopectin, agar, quince seed (Cydonia oblonga Mill), starch (rice, corn, potato, wheat), algae colloids (algae extract), microbiological polymers such as dextran, succinoglucan, starch-based polymers such as carboxymethyl starch, methylhydroxypropyl starch, alginic acid-based polymers such as sodium alginate, alginic acid propylene glycol esters, acrylate polymers such as sodium polyacrylate, polyacrylamide, polyethyleneimine, and inorganic water soluble material such as bentonite, aluminum magnesium silicate, laponite, hectonite, and anhydrous silicic acid.

Commercially available gelling agents useful herein include xanthan gum with tradename KELTROL series available from Kelco, Carbomers with tradenames CARBOPOL 934, CARBOPOL 940, CARBOPOL 950, CARBOPOL 980, and CARBOPOL 981, all available from B. F. Goodrich Company, acrylates/steareth-20 methacrylate copolymer with tradename ACRYSOL 22 available from Rohm and Hass, polyacrylamide with tradename SEPIGEL 305 available from Seppic, glyceryl polymethacrylate with tradename LUBRAGEL NP, and a mixture of glyceryl polymethacrylate, propylene glycol and PVM/MA copolymer with tradename LUBRAGEL OIL available from ISP, scleroglucan with tradename Clearogel SC11 available from Michel Mercier Products Inc. (NJ, USA), ethylene oxide and/or propylene oxide based polymers with tradenames CARBOWAX PEGs, POLYOX WASRs, and UCON FLUIDS, all supplied by Amerchol.

Useful herein are amphoteric polymers such as Polyquaternium 22 with tradenames MERQUAT 280, MERQUAT 295, Polyquaternium 39 with tradenames MERQUAT PLUS 3330, MERQUAT PLUS 3331, and Polyquaternium 47 with tradenames MERQUAT 2001, MERQUAT 2001N, all available from Calgon Corporation. Other useful amphoteric polymers include octylacrylamine/acrylates/butylaminoethyl methacrylate copolymers with the tradenames AMPHOMER, AMPHOMER SH701, AMPHOMER 28-4910, AMPHOMER LV71, and AMPHOMER LV47 supplied by National Starch & Chemical.

### First Pigment Component

The collapsible water-containing capsule of the present composition comprises a first pigment component having a particle size of less than 1 μm, preferably from about 5 nm to about 600 nm, more preferably from about 10 nm to about 500 nm. The first pigment component is inherently hydrophobic, or hydrophobically surface coated, preferably surface coated with a lipophobic hydrophobic coating material. For providing a preferred stable composition, the first pigment component is comprised, by weight of the capsule, from about 1% to about 29.9%, more preferably from about 5% to about 20%, of the capsule.

In one preferred embodiment, the first pigment component is surface coated with a lipophobic hydrophobic coating material. Without being bound by theory, in such preferred embodiment, it is believed that, by the surface tension of the lipophobic hydrophobic surface of the first pigment component, the first pigment component aligns at the phase boundary of the water phase, while the particles of the first pigment component bind with each other via van-der-Waals binding. Hence, the first pigment component covers the water phase. It is further believed that the overall structure due to the lipophobic hydrophobic surface, combined with the relatively small particle size of the first pigment component, contributes to the suitable shear stress tolerance of the collapsible water-containing capsule of the present composition.

The base pigments of the first pigment component useful herein include those that provide color or change tone, and also those that provide a certain skin feel. Useful pigments herein include clay mineral powders such as talc, magnesium silicate, synthetic fluorphlogopite, calcium silicate, aluminum silicate, silicate, bentonite and montomorilonite. The coloring powders useful herein include pearl pigments such as alumina, barium sulfate, calcium secondary phosphate, zirconium oxide, zinc oxide, hydroxy apatite, iron oxide, iron titate, ultramarine blue, Prussian blue, chromium oxide, chromium hydroxide, cobalt oxide, cobalt titanate, titanium oxide coated mica; organic powders such as polyester, polyethylene, polypropylene, polystyrene, methyl metharylate copolymer, cross-linked polymethyl methacrylate, 12-nylon, 6-nylon, polystyrene, styrene-acrylic acid copolymers, vinyl chloride polymer, vinyl acetates, tetrafluoroethylene polymer, polyorganosilsesquioxane resins, solid silicone elastomers, boron nitride, fish scale guanine, laked tar color dyes, and laked natural color dyes. Particularly useful herein as the first pigment component are titanium dioxide, zinc oxide, iron oxide, barium sulfate, polystyrene, silicate, and mixtures thereof.

When the first pigment component is not inherently hydrophobic, the surface is coated with materials having hydrophobic characteristics. Useful hydrophobic coating materials herein include organic silicone compounds, metal soap, oils, and material having both lipophobic and hydrophobic characteristics, such as fluorine compounds. Particularly suitable fluorine compounds herein are selected from the group consisting of perfluorooctyl triethoxylsilane, perfluoroalkylphosphoric acids, their salts, and mixtures thereof.

Commercially available first pigment components highly useful herein include Titanium Dioxide coated with C9-15 fluoroalcohol phosphates (0.25 μm) with tradename PF-5 TiO2 CR-50, Titanium Dioxide coated with perfluorooctyl triethoxysilane (0.021 μm) with tradename FHS-12 TiO2 P-25, Zinc Oxide coated with C9-15 fluoroalcohol phosphates (0.020 μm) with tradename PF-7 ZnO-350, Yellow Iron Oxide coated with C9-15 fluoroalcohol phosphates (0.435 μm) with tradename PF-5 YELLOW LL-100PD, Red Iron Oxide coated with C9-15 fluoroalcohol phosphates (0.44 μm) with tradename PF-5 RED R-516PD, and Black Iron Oxide coated with C9-15 fluoroalcohol phosphates (0.4 μm) with tradename PF-5 BLACK BL-100P PF-5 BLACK BL-100P PF-5 BLACK BL-100P, all available from Daito Kasei, hydrophobic silicates with tradename AEROSIL RY200S, AEROSIL R202, AEROSIL 805, AEROSIL R812S, AEROSIL 917, AEROSIL 974, AEROSIL 972, AEROSIL RX200, AEROSIL RX300, all available from Nippon Aerosil, and CAB-O-SIL TS530, CAB-O-SIL TS720, all available from Cabot Corporation

### Second Pigment Component

In one preferred embodiment, the collapsible water-containing capsule of the present composition further comprises, by weight of the capsule, from about 0.1% to about 29%, preferably from about 1% to about 10%, of a second pigment component. The second pigment component herein has a particle size of 1 μm or more, preferably from about 1 μm to about 25 μm, more preferably from about 4 μm to about 15 μm, is surface coated with a hydrophobic coating material. Pigments having spherical shape are preferred. Without being bound by theory, it is believed that, by the larger size of the second pigment component, the second pigment component aligns at the phase boundary of the first pigment component. It is believed that the dual covered structure provided by the first and second pigment components provide the suitable shear stress tolerance of the collapsible water-containing capsule of the present composition. It is believed that pigments having spherical shape are advantageous.

When included, the total of the first pigment component and the second pigment component is at least about 8% of the capsule, preferably from about 8% to about 25% of the capsule.

When included, the second pigment component also provides a unique appearance effect or skin feel that is not easily delivered by the first pigment component. In one example, the first pigment components alone may provide an overly matte finish and emphasize, rather than hide, skin unevenness such as pores. A spherical and translucent second pigment component can improve the natural appearance by light diffusion effect due to its shape and translucency. In another example, the first pigment components alone may provide a squeaky feel on the skin due to their small size. A soft spherical second pigment component may alleviate such negative skin feel and provide good smooth feel.

The base pigments of the second pigment component useful herein include; polyacrylates, silicates, sulfates, alumina, metal dioxides, carbonates, celluloses, polyalkylenes, vinyl acetates, polystyrenes, polyamides, acrylic acid ethers, silicones, mica, and mixtures and complexes thereof. Specifically, materials useful herein include polyacrylates such as methyl methacrylate copolymer and nylon, cross linked polymethyl methacrylate; silicates such as calcium silicate, magnesium silicate, barium silicate, aluminium silicate and silica beads; alumina; metal dioxides such as titanium dioxide and aluminium hydroxide; carbonates such as calcium carbonate, magnesium carbonate; celluloses; polyalkylenes such as polyethylene, and polypropylene; vinyl acetates; polystyrenes; polyamides; acrylic acid ethers such as acrylic acid methyl ether and acrylic acid ethyl ether; polyvinyl pyrrolidones; and silicones such as polyorganosilsesquioxane resin and solid silicone elastomers. Highly preferred materials are polymethyl methacylate.

In one embodiment, polyorganosilsesquioxane resin and solid silicone elastomers may be used for enhancing the effect of hiding skin pores.

The second pigment component herein is surface coated with a coating material having hydrophobic characteristics, whereby lipophobic hydrophobic coating materials are preferred. Useful hydrophobic coating materials herein include methyl polysiloxane, methyl hydrogen polysiloxane, methyl phenyl polysilxoane, n-octyl triethoxy silane, methyl-alpha-styrene polysiloxane, acryl silicone copolymer, and mixtures thereof. Preferred lipophobic hydrophobic coating materials are the same coating material as aforementioned for the first pigment component.

Commercially available second pigment components highly useful herein include mica (10 μm) with tradename SERICITE FSE available from Sanshin Kohkoh. Commercially available spherical second pigment components highly useful herein include methyl methacylate copolymer with tradename GANZ PEARL series available from Ganz Chemical Co., Ltd., and SYLYSIA series available from Fuji Sylysia Chemical, Nylon-12 with tradename NYLON POWDER series available from Toray Dow Corning, Nylon-12 coated with C9-15 fluoroalcohol phosphates (5 μm) with tradename PF-5 NYLON SP 500 available from Daito Kasei, polymethyl silsesquioxiane coated with with C9-15 fluoroalcohol phosphates with tradename PF-5 TOSPEARL 145 available from Daito Kasei, vinyl dimethicone/methicone silsesquioxane crosspolymer with tradenames KSP series available from ShinEtsu Chemical Co., Ltd., Tokyo Japan, and hardened polyorgano siloxane elastomers with tradenames TREFIL series available from Toray Dow Corning.

### Additional Components

The capsules hereof may further contain additional components such as are conventionally used in topical products, e.g., for providing aesthetic or functional benefit to the composition or personal surface, such as sensory benefits relating to appearance, smell, or feel, therapeutic benefits, or prophylactic benefits (it is to be understood that the above-described required materials may themselves provide such benefits). Further, the capsule of the present invention may comprise various skin benefit agents and perfumes in a dissolved or dispersed form in the water phase or attracted within the pigment components. It is advantageous to deliver such skin benefit agents, and perfumes encompassed in the present collapsible water-containing capsule, for one or more reasons. For those components that are heat sensitive, the present capsule prevents or delays evaporation prior to use. For those components that may be deteriorated or compromised in benefit by coming to contact with the remainder of the personal care composition, the present capsules act as a barrier. Other components may provide a certain sensation upon application and collapsing of the present capsule. When included, the total amount of additional components are kept to no more than about 10% by weight of the capsule.

Examples of suitable topical ingredient classes include: powders and pigments that do not meet the definition of pigment components described above, skin lightening agents, anti-acne agents, emollients, non-steroidal anti-inflammatory agents, topical anaesthetics, artificial tanning agents, antiseptics, anti-microbial and anti-fungal actives, skin soothing agents, UV protection agents, skin barrier repair agents, anti-wrinkle agents, anti-skin atrophy actives, lipids, sebum inhibitors, sebum inhibitors, skin sensates, protease inhibitors, skin tightening agents, anti-itch agents, hair growth inhibitors, desquamation enzyme enhancers, anti-glycation agents, antiperspirant actives, oxidative hair colorants, hair styling agents, anti-chelating agents, abrasives, astringents, dyes, essential oils, fragrance, film forming polymers, solubilizing agents, anti-caking agents, antifoaming agents, binders, buffering agents, bulking agents, denaturants, pH adjusters, propellants, reducing agents, sequestrants, cosmetic biocides, and preservatives.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

The following are products comprising a capsule composition and an applicator for use on the skin, method of preparation thereof, method of use, and assessment of their characteristics thereof. Examples 1-4 are capsule compositions according to the present invention. Applicator A is an applicator according to the present invention, while Applicator B is an applicator that is not according to the present invention.

TABLE 1
Composition for Examples 1-4

| | | Ex. 1 | Ex. 2 | Ex. 3 | Ex. 4 |
|---|---|---|---|---|---|
| 1 | Titanium Dioxide coated with C9-15 | 10 | | | 3 |
| | fluoroalcohol phosphates (0.25 μm) *1 | | | | |
| 2 | Titanium Dioxide coated with perfluorooctyl | | 10 | | |
| | triethoxysilane (0.021 μm) *2 | | | | |
| 3 | Zinc Oxide coated with C9-15 fluoroalcohol | | | | 2 |
| | phosphates (0.020 μm) *3 | | | | |
| 4 | Yellow Iron Oxide coated with C9-15 | 1.386 | | | 1.0 |
| | fluoroalcohol phosphates (0.435 μm) *4 | | | | |
| 5 | Red Iron Oxide coated with C9-15 fluoroalcohol | 0.189 | | | 0.1 |
| | phosphates (0.44 μm) *5 | | | | |
| 6 | Black Iron Oxide coated with C9-15 | 0.243 | | | 0.1 |
| | fluoroalcohol phosphates (0.4 μm) *6 | | | | |
| 7 | Anhydrous Silicic Acid coated with | | | 5 | |
| | hexamethylenedisilazane (7 nm) *7 | | | | |
| 8 | Anhydrous Silicic Acid coated with trimethyl | | | | 10 |
| | silyl (14 nm) *8 | | | | |
| 9 | Sodium Carboxymethyl Starch *9 | 1.5 | 1.5 | | 1.5 |
| 10 | Xanthan Gum *10 | | | 0.01 | 0.01 |
| 11 | Agar *11 | | | 0.5 | |
| 12 | Nylon-12 coated with C9-15 fluoroalcohol | 6.682 | 7.7 | | |
| | phosphates (5 μm) *12 | | | | |
| 13 | Polymethyl Silsesquioxiane coated with C9-15 | | | | 1 |
| | fluoroalcohol phosphates (4.5 μm) *13 | | | | |
| 14 | Talc coated by Methicone (10 μm) *14 | | | | 5.0 |
| 15 | Mica coated with Titanium Dioxide coated with | | | 12 | |
| | Methicone (40 μm) *15 | | | | |
| 16 | Mica (10 μm) *16 | | 1.85 | | |
| 17 | Titanium Dioxide (0.040 μm) *17 | | | | 1 |
| 18 | Polyoxyethylene Methylpolysiloxane | | | 0.5 | |
| | Copolymer *18 | | | | |
| 19 | D-delta-tocopherol *19 | | 0.1 | | |
| 20 | Ethylhexyl Methoxycinnamate *20 | | | 0.1 | |
| 21 | Butylene Glycol *21 | 15 | | 10 | |
| 22 | Dipropylene Glycol | | | | 10 |
| 23 | Glycerin | | 5 | 1 | |
| 24 | Ethanol | | 2 | | |
| 25 | Glucosyl Hesperidin *22 | | 0.5 | | |
| 26 | Ascorbic Acid *23 | | 1 | | |
| 27 | Niacinamide *24 | | 2 | | |
| 28 | Mulberry Root Extract *25 | | | | 1 |
| 29 | Panthenol *26 | | 1 | | |
| 30 | Saccharomycopsis Ferment Filtrate *27 | | 10 | | 5 |
| 31 | WATER | 64.7 | 56.9 | 70.59 | 58.89 |
| 32 | EDTA-2NA | | 0.1 | | 0.1 |
| 33 | PRESERVATIVES | 0.3 | 0.3 | 0.3 | 0.3 |
| 34 | Perfume | | 0.05 | | |
| | Total | 100 | 100 | 100 | 100 |

Definitions of Components
*1 Titanium Dioxide coated with C9-15 fluoroalcohol phosphates (0.25 μm): PF-5 TiO2 CR-50 available from Daito Kasei.
*2 Titanium Dioxide coated with perfluorooctyl triethoxysilane (0.021 μm): FHS-12 TiO2 P-25 available from Daito Kasei.
*3 Zinc Oxide coated with C9-15 fluoroalcohol phosphates (0.020 μm): PF-7 ZnO-350 available from Daito Kasei.
*4 Yellow Iron Oxide coated with C9-15 fluoroalcohol phosphates (0.435 μm): PF-5 YELLOW LL-100PD available from Daito Kasei.
*5 Red Iron Oxide coated with C9-15 fluoroalcohol phosphates (0.44 μm): PF-5 RED R-516PD available from Daito Kasei.
*6 Black Iron Oxide coated with C9-15 fluoroalcohol phosphates (0.4 μm): PF-5 BLACK BL-100P available from Daito Kasei.
*7 Anhydrous Silicic Acid coated with hexamethylenedisilazane (7 nm): AEROSIL RX300 from NIPPON AEROSIL.
*8 Anhydrous Silicic Acid coated with trimethyl silyl (14 nm): AEROSIL R202 from NIPPON AEROSIL.
*9 Sodium Carboxymethyl Starch: COVAGEL available from LCW.
*10 Xanthan Gum: Keltrol T available from Kelco.
*11 Agar: INA AGAR from Ina Food.
*12 Nylon-12 coated with C9-15 fluoroalcohol phosphates (5 μm): PF-5 NYLON SP-500 available from Daito Kasei.
*13 Polymethyl Silsesquioxiane coated with C9-15 fluoroalcohol phosphates (4.5 μm): PF-5 TOSPEARL 145 available from Daito Kasei.
*14 Talc coated by Methicone (10 μm): SI TALC from Miyoshi Kasei.
*15 Mica coated with Titanium Dioxide coated with Methicone (40 μm): SI FLAMENCO SUPER PEARL from Miyoshi Kasei
*16 Mica (10 μm): SERICITE FSE available from Sanshin Kohkoh.
*17 Titanium Dioxide (0.040 μm): Titanium Dioxide TTO-55 available from Ishihara.
*18 Polyoxyethylene Methylpolysiloxane Copolymer: KF-6018 from ShinEtsu Chemical.
*19 D-delta-tocopherol: D-DELTA-TOCOPHEROL available from EISAI CO., LTD.
*20 Ethylhexyl Methoxycinnamate: PARSOL MCX available from ROCHE VITAMINS JAPAN K.K.
*21 Butylene Glycol: 1,3-Butylene Glycol available from Celanese.
*22 Glucosyl Hesperidin: α-Ghesperidin PS-CC, available from Hayashibara.
*23 Ascorbic Acid: Ascorbic Acid available from ROCHE VITAMINS JAPAN K.K.
*24 Niacinamide: Niacinamide USP available from DSM.
*25 Mulberry Root Extract: Mulberry BG, available from Maruzen Pharmaceuticals.
*26 Panthenol: D-Panthenol USP, available from DSM.
*27 Saccharomycopsis Ferment Filtrate: SK-II Pitera available from Kashiwayama.

### Method of Preparation

The capsules of Examples 1-2 can be made as follows:

Components (1)-(13) are mixed and transferred to a container that has a hydrophobic inner surface. Components (21)-(33) are separately mixed and transferred to the same container. The container is closed and shacked by hands for a couple minutes. After naked eye observation of capsules in loose powder form, components (14)-(20) & (34) that had been separately mixed are transferred to the container and mixed.

The capsules of Examples 3-4 can be made as follows:

Components (9)-(11) and (21)-(33) are mixed with heating and transferred to a container that has a hydrophobic inner surface. Components (7)-(8) are added to the same container. Then they are mixed. After naked eye observation of capsules in loose powder form, components (1)-(6), (12)-(20) & (34) that had been separately mixed are transferred to the container and mixed.

### Applicators

Applicator A: HP-70 made of polyurethane provided by Bridgestone
Applicator B: Max Factor Profinish Buff made of NBR provided by Max Factor K.K

### Method of Use

Products comprising Composition 1 and Applicator A, as well as Composition 4 and Applicator A, are useful as foundation products. Products comprising Composition 2 and Applicator A are useful as whitening powders. Products comprising Composition 3 and Application A are useful as point make-up products. These products, when applied to the skin, provide good fit on the skin, favorable moisturizing and cooling sensation, even coverage, and long wear on the skin.

### Sensory Test

A sensory test using Applicators A and B detailed above for applying the composition of Example 1 detailed above to the facial skin was conducted. Example 1 was made by the process also detailed above. Both Applicators A and B were provided in a size commonly used for powder foundation products.

Twenty (20) Japanese females of age 25-49 who use foundation at least 5 times per week were divided into two groups of 10 panelists each. The first group used Example 1 with Applicator A for 2 days, then Example 1 with Applicator B for 2 days. The second group used Example 1 with Applicator B for 2 days, then Example 1 with Applicator A for two days. After total 4 days of usage, the panelists were asked 3 questions as described in Table 2, and evaluated the applicators as follows:
1. Applicator A was better than Applicator B
2. Applicator B was better than Applicator A
3. No difference between the applicators
   The percentage of panelists who favored Applicators A and B were calculated. Those panelists who provided answer 3 (no difference) were split into half and added to A and B for calculation purpose. The results were as shown in Table 2. Statistic analysis showed that Applicator A was significantly favored over Applicator B in all 3 questions.

TABLE 2
Sensory Test Results

| | A | B |
|---|---|---|
| Fits on Skin | 70 | 30 |
| Provides an even coverage | 73 | 27 |
| Feels moisturized | 65 | 35 |

As can be understood from the sensory test above, for applying the capsule ot the present invention to the skin, applicators made of material having open cell structure provide significantly better performance over applicators made of material having closed cell structure.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care product comprising:
(a) a collapsible water-containing capsule having water phase encapsulated or dispersed in a first pigment component having a particle size of less than 1 μm; and
(b) an applicator for applying the capsule on a personal care surface, wherein the surface of the applicator is made of material having open cells.

2. The personal care product of claim 1 wherein the applicator is made of polyurethane.

3. The personal care product of claim 2 wherein the material for the applicator is treated to remove foam films.

4. The personal care product of claim 1 wherein the applicator is made of material having open cells having an average cell range of from about 40/25 mm to about 80/25 mm.

5. The personal care product of claim 1 wherein the applicator further comprises raised projections, wherein despite the raised projections, the surface of the material having open cells serves as a temporary reservoir of the capsules.

6. The personal care product of claim 1 wherein the capsule is stable under normal storage conditions as well as normal mixing processes, however, collapses upon application.

7. The personal care product of claim 6 wherein the capsule is substantially free of porous pigments having a particle size of less than 1 μm.

8. The personal care product of claim 7 wherein the capsule comprises by weight:
(a) from about 70% to about 92% of a water phase comprising, by weight of the capsule:
(1) from about 5% to about 91.9% water; and
(2) from about 0.1% to about 20% of a gelling agent;
(b) from about 1% to about 29.9% of a first pigment component which has a particle size of less than 1 μm and is surface coated with a lipophobic hydrophobic coating material; and
(c) from about 0.1% to about 29% of a second pigment component which has a particle size of 1 μm or more, is surface coated with a hydrophobic coating material; and is spherical in shape; wherein the total of the first pigment component and the second pigment component is at least about 8% of the capsule.

9. A method of making up of the skin using the personal care product of claim 1 comprising the steps of:
(1) providing the collapsible water-containing capsule;
(2) shearing the collapsible water-containing capsule on the skin by the applicator to allow the collapsible water-containing capsule to collapse; whereby the components of the collapsible water-containing capsule are applied on the skin; and
(3) allowing the water to evaporate and/or be absorbed in the skin.

10. A method of making up of the skin using the personal care product of claim 8 comprising the steps of:
(1) providing the collapsible water-containing capsule;
(2) shearing the collapsible water-containing capsule on the skin by the applicator to allow the collapsible water-containing capsule to collapse; whereby the components are applied on the skin in the order of vicinity from the skin, water, the first pigment component, and the second pigment component; and
(3) allowing the water to evaporate and/or be absorbed in the skin.
